(19) United States
(12) Patent Application Publication
(10) Pub. No.: US 2022/0082267 A1
(43) Pub. Date: Mar. 17, 2022

(54) Cooking Pollutant Control Methods Devices and Systems

(71) Applicant: Oy Halton Group Ltd., Helsinki (FI)

(72) Inventors: Derek W. SCHROCK, Bowling Green, KY (US); Andrey V. LIVCHAK, Bowling Green, KY (US); Fuoad A. PARVIN, Hermitage, TN (US)

(73) Assignee: Oy Halton Group Ltd., Helsinki (FI)

(21) Appl. No.: 17/425,446

(22) PCT Filed: Feb. 5, 2020
(86) PCT No.: PCT/US2020/016738

§ 371 Date: Jul. 23, 2021

Related U.S. Application Data
(60) Provisional application No. 62/939,034, filed on Nov. 22, 2019.
(60) Provisional application No. 62/801,276, filed on Feb. 5, 2019.

(51) Int. Cl. F24C 15/20; F24F 8/192; F24F 8/158; A61L 9/20; A61L 9/014

(52) U.S. Cl. CPC F24C 15/2021; F24C 15/2035; F24F 8/192; F24F 2110/66; A61L 9/20; A61L 9/014; F24F 8/158

(57) ABSTRACT

A cooking fume mitigation system may have an exhaust hood configured to capture fumes from a cooking appliance, the exhaust hood conveying fumes to a particulate removal stage which conveys fumes to an odor removal stage. The system may also have an inlet volatile organic compound (VOC) sensor upstream of the odor removal stage and an outlet VOC sensor downstream of the odor removal stage. The odor removal stage may include a carbon filter or an ultraviolet light source. The particulate removal stage may include a pocket filter or an electrostatic precipitator filter. The system may also have a controller that receives signals from the inlet and outlet VOC sensors and uses the signals to generate data indicative of a remaining life of the carbon filter.

### CROSS-REFERENCE TO RELATED APPLICATIONS

This application claims the benefit of U.S. Provisional Application No. 62/801,276 filed Feb. 5, 2019 and U.S. Provisional Application No. 62/939,034 filed Nov. 22, 2019, which are herein incorporated by reference in their entirety.

### BACKGROUND

Exhaust hoods are used to remove air contaminants close to the source of generation located in a conditioned space. For example, one class of exhaust hood, kitchen exhaust hoods, creates suction zones directly above ranges, fryers, or other sources of air contamination. The exhaust stream from such applications often contain large quantities of particulates, particularly hydrocarbons such as oil droplets.

Organic substances in the form of vapors or particles can also be formed by many production processes within various industries. For example, they can be generated by preparation and use of lacquer and paint, cereal and feedstuff, metal and plastic, tar and asphalt, tanneries, incinerating plants, bio-gas plants, agriculture, and many food preparation processes.

Because of concerns about the environment and worker health, it is desirable to find economically attractive mechanisms for removing organic substances from air streams. Air purification is frequently performed by filtering the contaminated air in, for example, grease filters and carbon filters. Mechanical filters, however, are expensive in terms of maintenance manpower and pressure drop, which leads to high operating costs. Furthermore, filters cannot guarantee fulfillment of high hygienic requirements.

One technology that has been used for degrading organic particulates in effluent streams is the addition of ozone to the effluent stream. This can be accomplished by irradiating with ultraviolet light or using a corona discharge. A negative side effect of using corona discharge is the creation of nitrogen oxides (NOx).

### SUMMARY

A multistage filter receives fumes from cooking after capture by a hood. The fumes first pass through a grease filter which uses inertial principles to remove aerosolized grease from the fumes. A pocket filter removes most of the remaining particulates which are conveyed to a minipleat pleated HEPA-type filter to further remove particulates. An optional ultraviolet treatment stage then receives the output of the minipleat pleated HEPA-type filter whereupon the fumes pass through an activated carbon filter, primarily for odor removal. Upstream and downstream of the carbon filter may be volatile organic compound (VOC) sensors. A controller receives signals from the upstream and downstream VOC sensors and uses them to estimate the remaining life of the activated carbon filter. Note that the carbon filter may include stages that include a zeolite filter, a VOC filter, and an odor filter.

Objects and advantages of embodiments of the disclosed subject matter will become apparent from the following description when considered in conjunction with the accompanying drawings.

### BRIEF DESCRIPTION OF THE DRAWINGS

Embodiments will hereinafter be described in detail below with reference to the accompanying drawings, wherein like reference numerals represent like elements. The accompanying drawings have not necessarily been drawn to scale. Where applicable, some features may not be illustrated to assist in the description of underlying features.

FIG. 1 shows a general framework of a pollution control system according to embodiments of the disclosed subject matter.

FIG. 2 shows the general framework of a pollution control system with a fire control bypass element according to embodiments of the disclosed subject matter.

FIG. 3 shows the general framework of a pollution control system with a fire control bypass element and an odor sensor element according to embodiments of the disclosed subject matter.

FIG. 4 shows the general framework of a pollution control system with a fire control bypass element, an odor sensor element, and a detail of a first particulate module type according to embodiments of the disclosed subject matter.

FIG. 5A shows the general framework of a pollution control system with a fire control bypass element, an odor sensor element, and a detail of a second particulate module type according to embodiments of the disclosed subject matter.

FIG. 5B shows the general framework of a pollution control system with a fire control bypass element, an odor sensor element, and a detail of a third particulate module type according to embodiments of the disclosed subject matter.

FIG. 6 shows the general framework of a pollution control system with a fire control bypass element, an odor sensor element, and a detail of a fourth particulate module type according to embodiments of the disclosed subject matter.

FIG. 7 shows the general framework of a pollution control system with a fire control bypass element, an odor sensor element, and a detail of a first odor removal stage according to embodiments of the disclosed subject matter.

FIG. 8 shows the general framework of a pollution control system with a fire control bypass element, an odor sensor element, and a detail of a second odor removal stage according to embodiments of the disclosed subject matter.

FIG. 9 shows the general framework of a pollution control system with a fire control bypass element, an odor sensor element, and a detail of a third odor removal stage according to embodiments of the disclosed subject matter.

FIG. 10 shows the general framework of a pollution control system with a fire control bypass element, an odor sensor element, and a detail of a fourth odor removal stage according to embodiments of the disclosed subject matter.

FIG. 11 shows an embodiment that has no odor removal stage, according to embodiments the disclosed subject matter.

FIG. 12 shows a sampling device with separate sampling ports.

FIG. 13 shows a disclosure of a computer system that embodies elements of any controllers disclosed herein.

### DETAILED DESCRIPTION

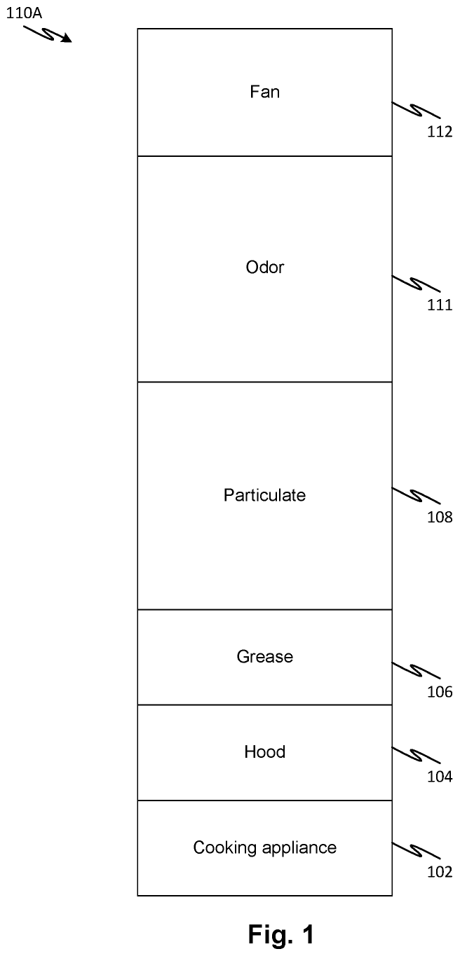

Referring to FIG. 1, an embodiment of a pollution control system for kitchens 110A receives fumes from a cooking appliance 102 via an exhaust hood 104. Grease filters 106 may capture grease particulates. The fumes from the grease filters 106 pass into a particulate removal stage 108 and an odor removal stage 111. The particulate removal stage 108 and the odor removal stage 111 may have various detailed embodiments according to the disclosed subject matter. A fan 112 finally draws the fumes through the entire system. As discussed below, the odor removal stage 111 may be configured responsively to various criteria such as the strength of the odor and the type. Similarly, the particulate removal stage 108 may be configured responsively to various criteria such as the particulate load.

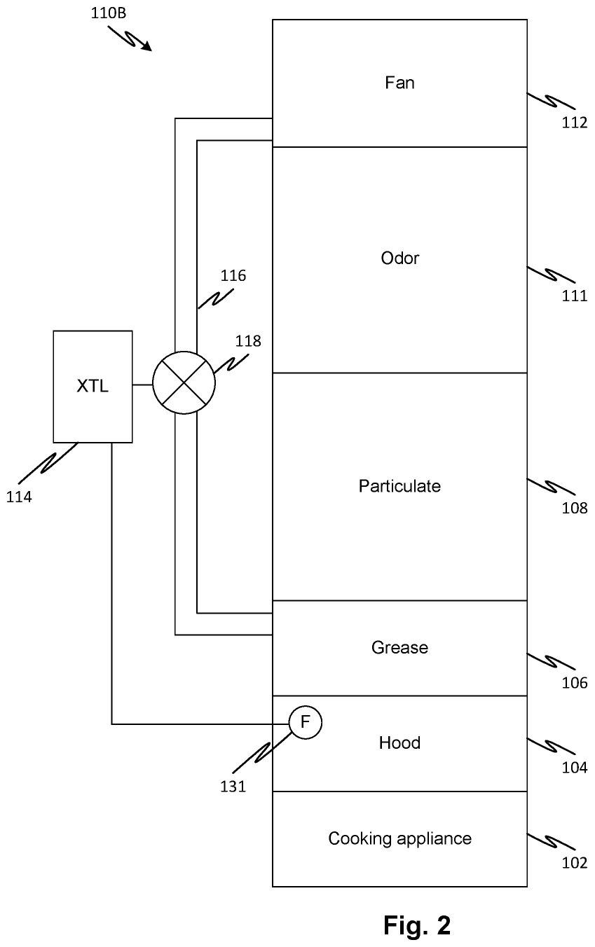

FIG. 2 shows an embodiment of a pollution control system for kitchens 110B with a fire control bypass element with a controller 114, a flow control bypass valve or damper 118, and a bypass duct 116. A fire sensor 131 applies a signal indicating a fire to the controller. In the event of a fire, the bypass valve or damper 118 redirects flow of fumes through the bypass duct 116 avoiding the conveyance of burning gases through the particulate removal stage 108 and odor removal stage 111. Although the bypass valve or damper 118 is shown as a single device, its depiction is symbolic. Persons of ordinary skill are able to design various configurations to perform the function of diverting flow from the grease filter 106 through the bypass duct 116 to the fan intake. Generally, a pair of two-way diverting dampers will be effective for this function with one two-way diverting damper at each end of the bypass duct.

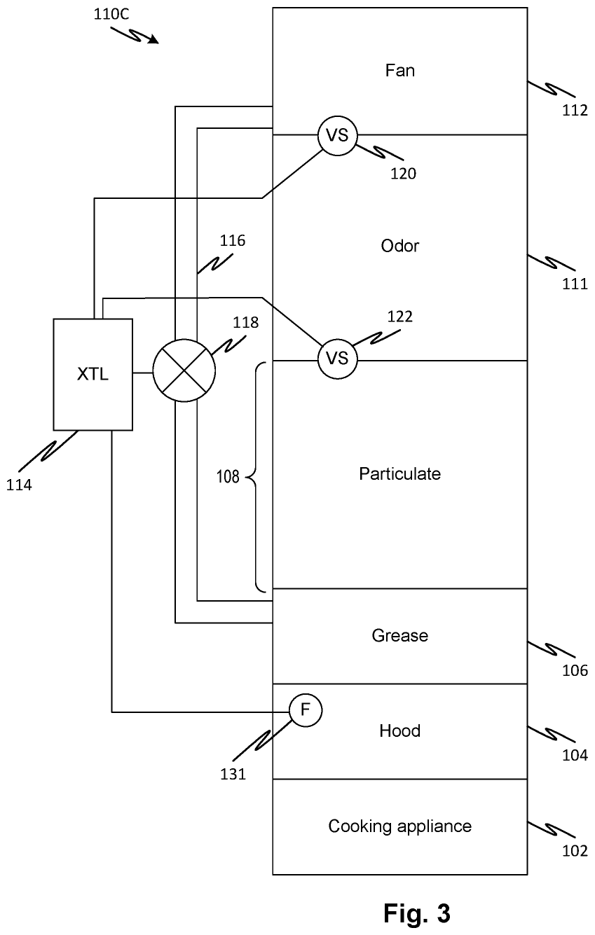

FIG. 3 shows an embodiment of a pollution control system for kitchens 110C with a fire control bypass element and an odor sensor element according to embodiments of the disclosed subject matter. The odor sensing element has two VOC sensors, an upstream VOC sensor 122 and a downstream VOC sensor 120. Signals from the upstream VOC sensor 122 and a downstream VOC sensor 120 are applied to the controller 114. Upstream and downstream are relative to the flow direction of fumes in the pollution control system. The separate upstream VOC sensor 122 and downstream VOC sensor 120 permit a comparison between the VOC concentration of the fumes passing into the odor removal stage 111 and those passing out of the odor removal stage 111. Such a comparison may reveal conditions of the odor removal stage 111 better than a single sensor on the outlet. For example, an expired odor control stage may exhibit higher VOC concentration from the odor removal stage 111 than entering it.

VOC or Volatile Organic Compounds are organic chemicals that have a high vapor pressure under normal atmospheric pressures and temperatures. As a result, they have a low boiling point and readily evaporate into the atmosphere. A VOC sensor, such as 120 and 122, measures the presence of VOCs and outputs a signal representing a value proportional to the amount of VOC detected, sometimes measured in parts per million.

VOCs in the fumes can be detected based on different principles and interactions between the organic compounds and the sensor components. The VOC sensors 120 and 122 may be photoionization detectors (PID), that use a bright ultraviolet light source to knock electrons out of the VOC molecule and measure these electrons, where the flow of the electrons indicates that VOC molecules are present at the sensor. VOC molecules are complex and easily broken down by high energy photons. Each specific type of VOC molecule has an ‘ionization potential’ (IP) value that represents the amount of energy necessary to liberate an electron; this value is measured in ‘electron volts’, or eV. PID sensors have a specified level of energy, also measured in eV, and in general, any compound with an IP value less than the sensor's eV rating will be ionized and detected. For example, with a 10.6 eV VOC sensor, the presence of benzene (IP=9.24 eV) will be detected, whereas molecules water vapor (IP=12.6 eV) will not be detected.

The difference between the downstream and upstream VOC sensors may be recorded over time to create a trend of the difference. The trend may be used to determine when the odor removal stage 111 has reached the limits of its capacity. For example, for an activated carbon filter, this may detect the limit of adsorptivity of the activated carbon filter. Alternatively, this arrangement could detect when the carbon filter starts off-gassing (gasses being released from the filter) due to materials other than VOC's causing the trapped VOC's to become entrained or released from the filter into the airstream (otherwise known as re-entrained). For example, water is such a material.

Another method of analyzing the time-based trend signal from the VOC sensor would be to evaluate the rate of change of the sensor to determine if the rate of change is increasing or decreasing. For a given cooking process, the rate of change has a predefined signature. If the performance of the odor removal stage changes it is anticipated that the rate of change will also change.

To estimate life left in the VOC filter (e.g., carbon filter 140), an efficiency parameter that indicates the efficiency may be calculated by the controller 114 based on the signals from the upstream 122 and downstream 120 VOC sensors. For example, when the parameter is at, or above, a first threshold (e.g. 30% efficiency), a first indicator signal may be output to indicate the condition of the filter. For example, a user interface may show a green light in response to the first indicator signal. When the efficiency falls below the first adjustable threshold a second warning signal may be output. The second warning signal may take the form of a low level warning such as a yellow light. If the efficiency falls between the first threshold and a second threshold (e.g., 10% efficiency) the indicator may output a third warning signal. The third warning signal may take the form of, for example, a red light to indicate the filter should be changed. The yellow light indicates to a user that the filter may expire soon and the red light may indicate that the filter is expired and that it has to be replaced or cleaned. The yellow light may indicate that the filter will need to be replaced soon. Thus, the two VOC sensors 120 and 122 may be used to predict failure of the carbon filter and also detect when the carbon filter has failed.

In other embodiments, the estimation of the remaining life of the filter can be achieved without a digital controller. For example, a signal from each of the sensors 120 and 122 may be provided as an analog signal, with the voltage value representative of the measure of the amount of VOCs detected at each sensing location, to a device. The device may be a circuit that includes at least one operational amplifier. Examples of such devices are adders (full adder, half adder) and subtractors (full subtractor, half subtractor). Other analog circuits can also be designed that effectively compare the voltage level between the two signal, and output a signal representative of the difference. The voltage level of this output signal can be used to represent the remaining life of the filter. The level can be calibrated and then further compared to threshold levels so that an estimate of the remaining life can be made.

Note that any number or thresholds may be employed along with any type of output for indicating state of a VOC filter. For example, the life of the filter may be displayed as a numerical value indicating the time remaining (such as months, weeks, or days remaining) before a replacement is needed, so that a user can obtain the necessary replacement filter in time to perform the replacement. In other embodiments, the display may indicate a percentage value that decreases from 100 to 0 as indication of the remaining life of the filter, based on the calculations performed based on the output of sensors 120 and 122.

The threshold parameter may be, or be indicative of, an average of the instantaneous efficiency over a predefined interval, for example, a day or a shorter or longer interval. Alternatively, the efficiency parameter may be indicative of a maximum value of the measured efficiency over a predefined interval, for example, a day or a shorter or longer interval.

Note that the efficiency parameter may indicate a negative efficiency under some conditions in the described application where the filter is used to remove VOCs from a cooking application. Note that systems that only detect the VOC concentration downstream of the VOC filter cannot measure efficiency and further cannot detect a negative efficiency. Negative efficiency may occur when the filter is outgassing and indicates a condition where the filter should be replaced. Also, the upstream VOC sensor and downstream VOC sensor can be used by the controller to eliminate the effects of temperature and humidity.

The disclosed embodiments, by relying on two VOC sensors, or upstream and downstream sampling locations, provide an improvement in the ability to monitor the breakdown process of the filter and predict when a filter failure will occur. In embodiments, the system calculates a parameter with example thresholds at 30% (of predicted remaining life span of the filter) for yellow condition of the carbon filter, and 10% triggering the red condition. These threshold values are examples and are not limiting.

The disclosed embodiments allow the prediction of a filter failure before the actual failure. An example of a filter failure is filter breakthrough or the depletion of the carbon filter.

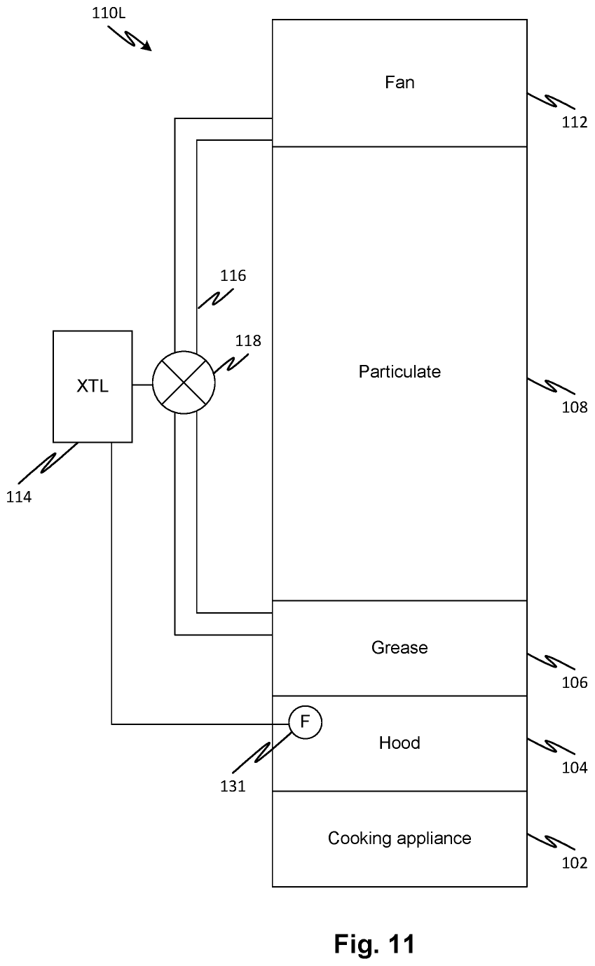

Referring to FIG. 12, in alternative embodiments a VOC sensor 120, 122 pair may be replaced by a sampling device 150 with a single VOC sensor 121 having a pair of sampling inlets. In this way the inlet fumes can be sampled and sent to the single VOC sensor 121 alternatingly, at different times. Sample times can be several seconds or longer. This alternative may be used in any of the embodiments. An advantage of the use of a single VOC sensor 121 with multiple sampling inlets is that it eliminates error due to differences between individual sensor responses. The sampling device 150 may include a flow switch and an air pump (not shown) to alternately convey air from a tube 172 before (i.e., upstream) the odor removal stage 111 to the single VOC sensor 121 and from a tube 171 after (i.e., downstream) the odor removal stage 111.

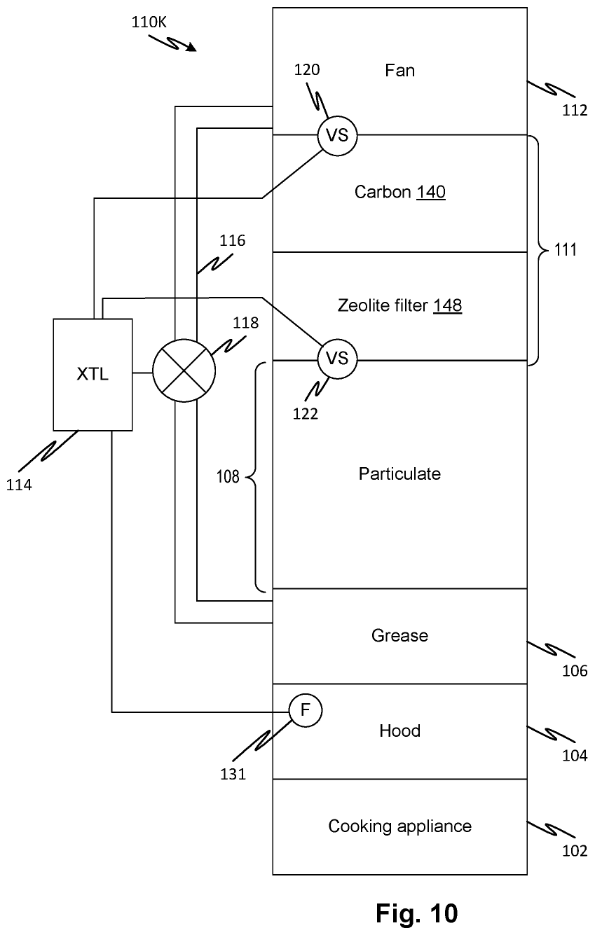

Another embodiment is one in which no odor removal stage 111 is used, as illustrated in FIG. 11. In such embodiments, the particulate removal stage 108 may take the form of any of the various detailed embodiments disclosed herein.

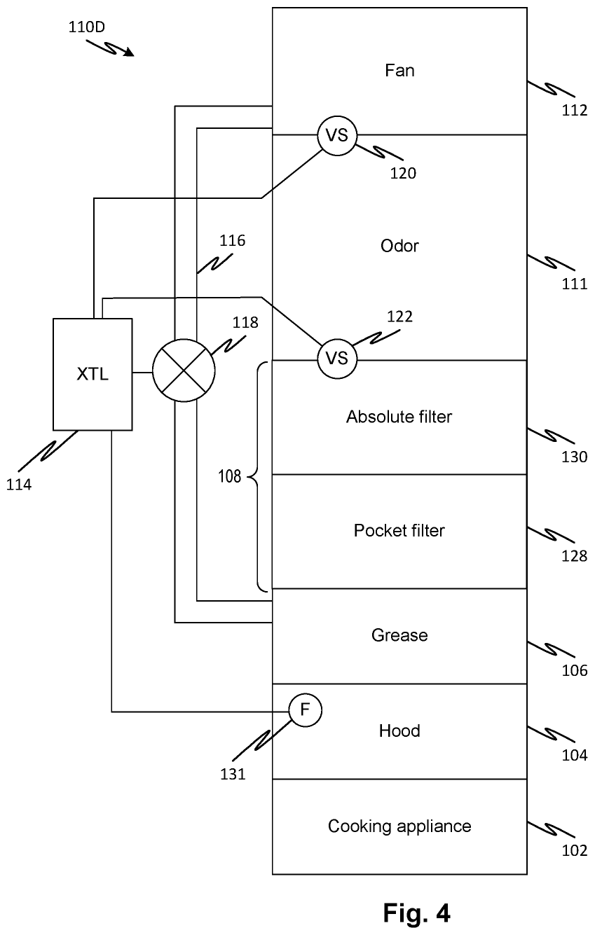

FIG. 4 shows an embodiment of a pollution control system for kitchens 110D with a fire control bypass element, an odor sensor element, and a detail of a first particulate module type according to embodiments of the disclosed subject matter. In the present embodiment, a detailed embodiment of the particulate removal stage 108, which may be used with any embodiment of the odor removal stage 111, has a pocket filter 128 and an absolute filter 130. The pocket filter may be as described in International Patent Publication WO2017062926, (incorporated herein by reference in its entirety) according to embodiments. An absolute filter 130 may be replaced with a High-efficiency particulate air (HEPA) filter. The pocket filter 128 may be replaced with a high capacity depth-loading filter, according to alternative embodiments.

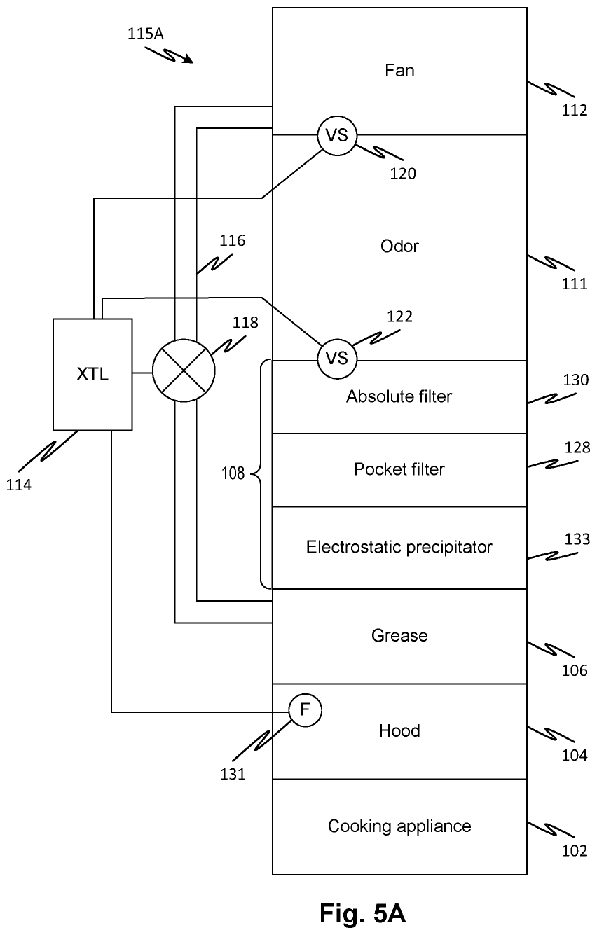

FIG. 5A shows an embodiment of a pollution control system for kitchens 115A with a fire control bypass element, an odor sensor element, and a detail of a particulate module with a single electrostatic precipitator according to embodiments of the disclosed subject matter. Such embodiments are suited to griddle cooking appliances or gas-fired grills, for example. The configuration retains the detail of the pocket filter 128 and absolute filter 130 from the previous embodiments.

FIG. 5B shows an embodiment of a pollution control system for kitchens 115B with a fire control bypass element, an odor sensor element, and a detail of a particulate module which is the same as the previous embodiment but with two electrostatic precipitators 135A and 135B rather than one, according to embodiments of the disclosed subject matter. Such an embodiment with two electrostatic precipitators would be suited to a heavy particulate load, for example, a cooking appliance that uses solid fuel for cooking, for example, wood fire or charcoal.

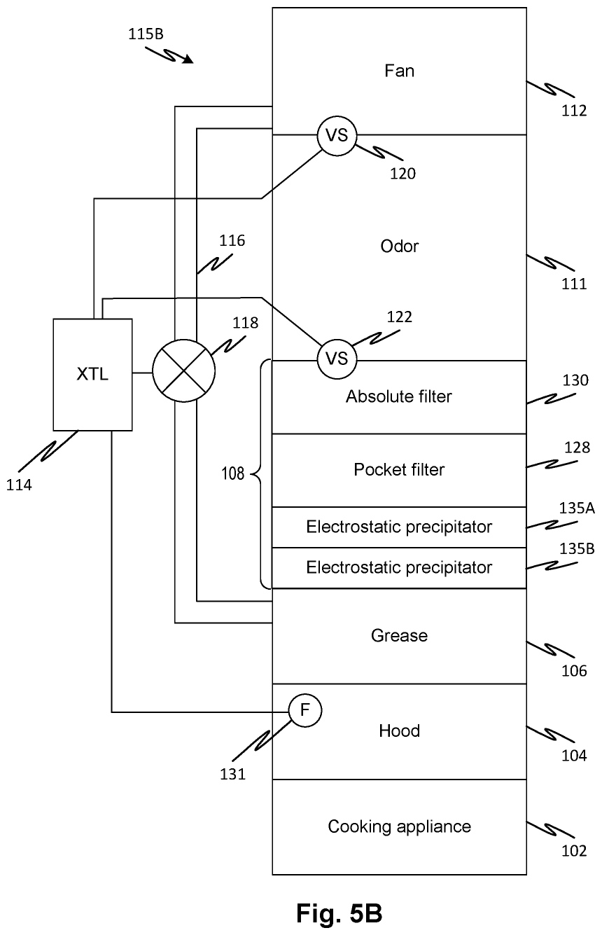

FIG. 6 shows an embodiment of a pollution control system for kitchens 110F with a fire control bypass element, an odor sensor element, and a detail of a fourth particulate module type according to embodiments of the disclosed subject matter. In the present embodiment, the particulate removal stage 108 has only an electrostatic precipitator 133 for particulate control.

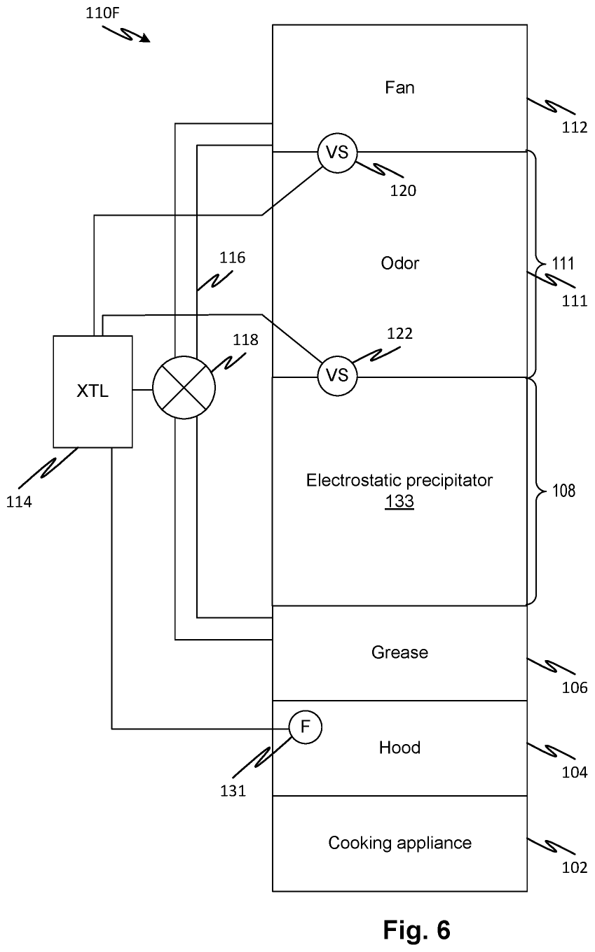

FIG. 7 shows an embodiment of a pollution control system for kitchens 110G with a fire control bypass element, an odor sensor element, and a detail of a first odor removal stage 111 according to embodiments of the disclosed subject matter. Here the carbon filter 140 is an activated charcoal filter. The upstream VOC sensor 122 and a downstream VOC sensor 120 are used to monitor the capacity of the charcoal filter 140 as described above.

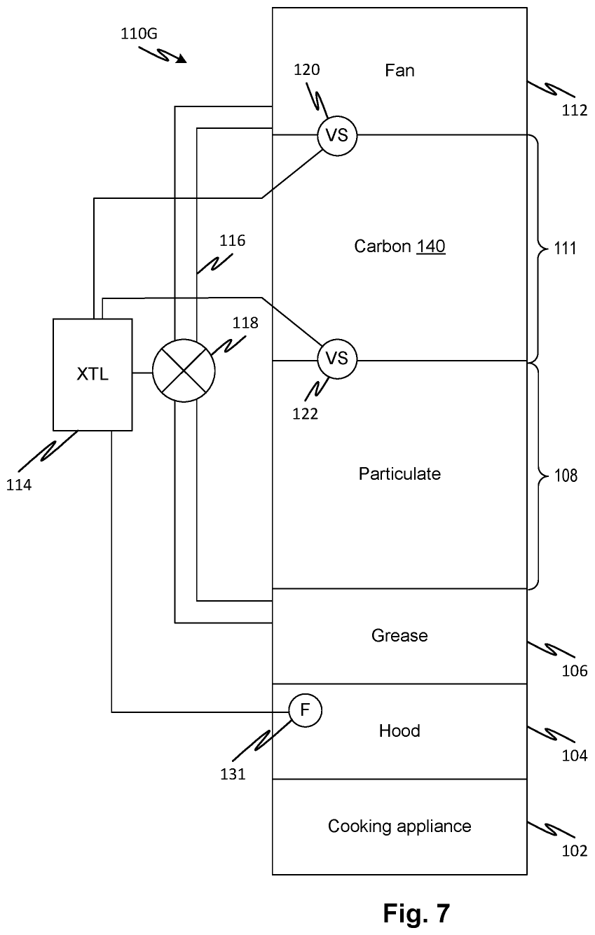

FIG. 8 an embodiment of a pollution control system for kitchens 110H with a fire control bypass element, an odor sensor element, and a detail of a second odor removal stage 111 according to embodiments of the disclosed subject matter. Here an odor spray 142 is used for the odor removal stage 111. The spray may be an odor masking agent or odor eliminator that is sprayed into the fume stream.

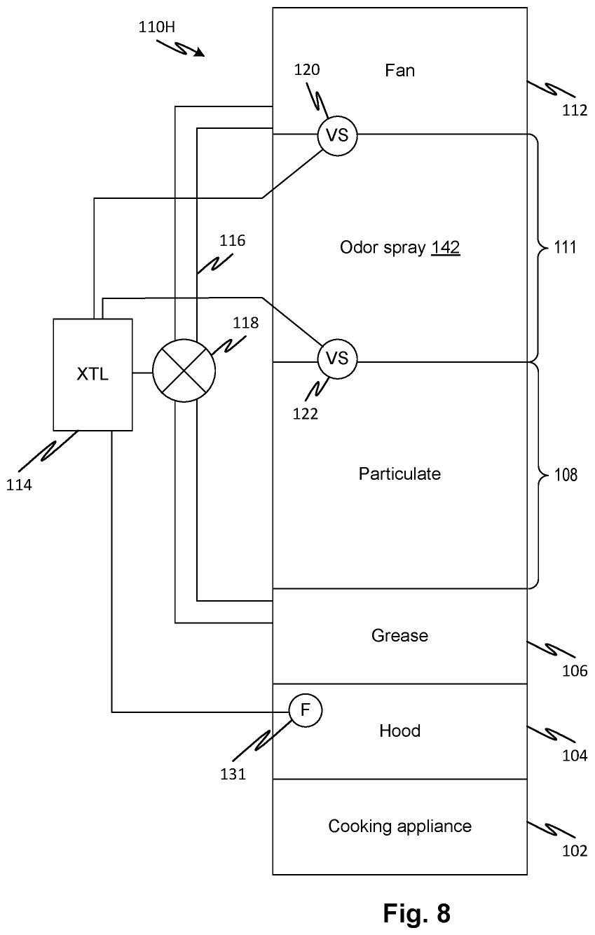

FIG. 9 shows an embodiment of a pollution control system for kitchens 110J with a fire control bypass element, an odor sensor element, and a detail of a third odor removal stage 111 according to embodiments of the disclosed subject matter. The odor removal stage 111 has a carbon filter 140 preceded by an ultraviolet filter 146.

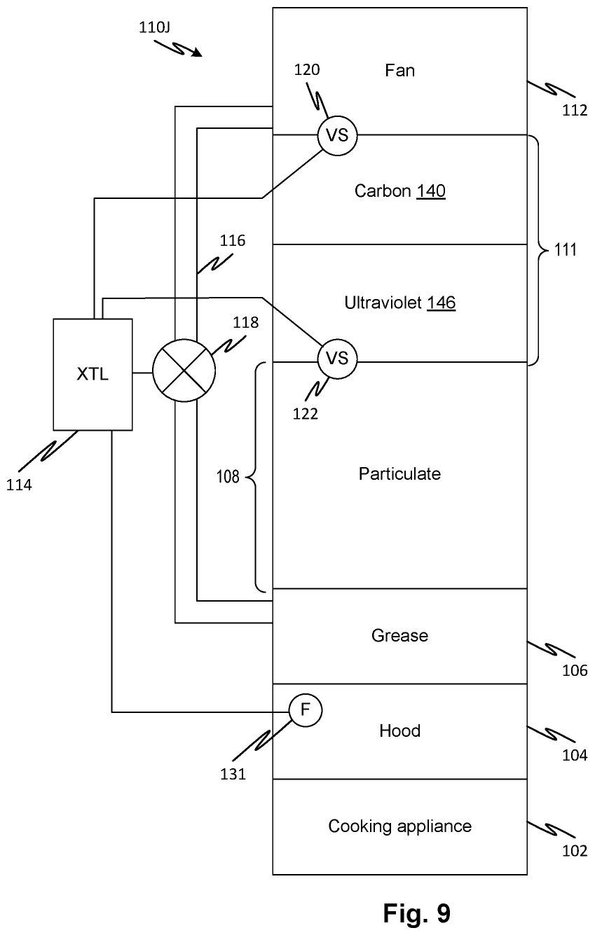

FIG. 10 shows an embodiment of a pollution control system for kitchens 110K a fire control bypass element, an odor sensor element, and a detail of a third odor removal stage 111 according to embodiments of the disclosed subject matter. Here the carbon filter 140 is preceded by a zeolite filter 148. Both have odor removal properties.

FIG. 11 shows an embodiment of a pollution control system for kitchens 110L with a fire control bypass element, an odor sensor element, and a particulate removal stage 108 according to embodiments of the disclosed subject matter. Here, no odor removal stage 111 is provided. For example, applications for embodiment this would be situations where odor control is not very important.

Note that any embodiment of a particulate control element can be combined with any odor control element.

Note that in embodiments with stringent odor control requirements or strong odor, the carbon filter may include multiple filter elements. These multiple filter elements would be changed on a rotating basis with the most upstream one removed and the others moved up in rank (in the upstream direction) and the one furthest downstream would be replaced.

According to embodiments, the disclosed subject matter includes a cooking fume mitigation system. An exhaust hood is configured to capture fumes from a cooking appliance. The exhaust hood conveys fumes to a particulate removal stage which conveys fumes to an odor removal stage. An inlet VOC sensor 122 is upstream of the odor removal stage and an outlet VOC sensor 120 is downstream of the odor removal stage.

In variations of the embodiments, the odor removal stage includes a carbon filter.

In variations of the embodiments, the odor removal stage also includes an ultraviolet light source.

In variations of the embodiments, the particulate removal stage includes a pocket filter.

In variations of the embodiments, the particulate removal stage includes an electrostatic precipitator filter.

In variations thereof, the embodiments include a controller that receives signals from the inlet and outlet VOC sensors and uses the signals to generate an estimate of the remaining life of the carbon filter.

According to embodiments, the disclosed subject matter includes a cooking fume mitigation system. An exhaust channel conveys fumes from a cooking appliance to a particulate removal stage which conveys fumes to an odor removal stage. An inlet VOC sensor 122 is upstream of the odor removal stage and an outlet VOC sensor 120 is downstream of the odor removal stage. In variations of the embodiments, the odor removal stage includes a carbon filter.

In variations of the embodiments, the odor removal stage also includes an ultraviolet light source.

In variations of the embodiments, the particulate removal stage includes a pocket filter.

In variations of the embodiments, the particulate removal stage includes an electrostatic precipitator filter.

In variations thereof, the embodiments include a controller that receives signals from the inlet and outlet VOC sensors and uses the signals to generate an estimate of the remaining life of the carbon filter.

According to embodiments, the disclosed subject matter includes an odor removal filter having a VOC sensor with a sampling device 150 having first and second sampling ports configured to convey samples of fumes from upstream and downstream of the odor removal filter to the VOC sensor.

In variations of the embodiments, the sampling device 150 conveys the samples intermittently to a single VOC sensor in order to obtain signals from different locations along the exhaust pathway from a same VOC sensor.

Variations of the embodiments include a controller that receives signals from the inlet and outlet VOC sensors and uses the signals to generate data indicative of an estimated remaining life of the carbon filter.

In variations of the embodiments, the controller is configured to calculate a parameter that depends on an efficiency of the odor removal stage.

In variations of the embodiments, the controller is configured to estimate a remaining life of a filter responsively to said parameter.

In variations of the embodiments, the controller is configured for calculating a parameter dependent on a negative efficiency and to use said parameter to control a signal output.

In variations of the embodiments, the controller outputs a medium level and a high level alert responsively to the parameter, the high level alert corresponding to a lower efficiency than the medium level alert.

In variations of the embodiments, the parameter related to efficiency is calculated once each day from a peak signal follower or an average of the parameter values over the course of the day.

In variations of the embodiments, the parameter related to efficiency is calculated once per a time interval from a peak signal follower (value following a peak signal) or an average of the parameter values over the course of the interval.

Embodiments of an odor removal device include an odor removal filter having a VOC sensor with a sampling device having first and second sampling ports configured to convey samples of fumes from upstream and downstream of the odor removal filter to the VOC sensor.

In variations of the embodiments, the sampling device conveys the samples intermittently to a single VOC sensor in order to obtain signals from different locations from a same VOC sensor.

Embodiments include a method of estimating a remaining life of a filter, include using a controller sampling a sensor signal upstream and downstream of a filter; using a controller, taking a maximum or average of said sensor signal over a course of a time interval and calculating a remaining life of said filter responsively to a parameter related to an efficiency of the filter. The estimation is based upon threshold values of said parameter where a high efficiency corresponds to a longer remaining life than a low efficiency.

In variations of the embodiments, the high efficiency is above 30% and the low efficiency is below or equal to 30%.

In variations of the embodiments, the filter is a carbon adsorption filter.

In variations of the embodiments, the filter is an adsorbent bed.

It will be appreciated that the modules, processes, systems, and sections described above can be implemented in hardware, hardware programmed by software, software instruction stored on a non-transitory computer readable medium or a combination of the above. For example, a method for controlling cooking fumes and odors can be implemented, for example, using a processor configured to execute a sequence of programmed instructions stored on a non-transitory computer readable medium. For example, the processor can include, but not be limited to, a personal computer or workstation or other such computing system that includes a processor, microprocessor, microcontroller device, or is comprised of control logic including integrated circuits such as, for example, an Application Specific Integrated Circuit (ASIC). The instructions can be compiled from source code instructions provided in accordance with a programming language such as Java, C++, C#.net or the like. The instructions can also comprise code and data objects provided in accordance with, for example, the Visual Basic™ language, LabVIEW, or another structured or object-oriented programming language. The sequence of programmed instructions and data associated therewith can be stored in a non-transitory computer-readable medium such as a computer memory or storage device which may be any suitable memory apparatus, such as, but not limited to read-only memory (ROM), programmable read-only memory (PROM), electrically erasable programmable read-only memory (EEPROM), random-access memory (RAM), flash memory, disk drive and the like.

Furthermore, the modules, processes, systems, and sections can be implemented as a single processor or as a distributed processor. Further, it should be appreciated that the steps mentioned above may be performed on a single or distributed processor (single and/or multi-core). Also, the processes, modules, and sub-modules described in the various figures of and for embodiments above may be distributed across multiple computers or systems or may be co-located in a single processor or system. Exemplary structural embodiment alternatives suitable for implementing the modules, sections, systems, means, or processes described herein are provided below.

The modules, processors or systems described above can be implemented as a programmed general purpose computer, an electronic device programmed with microcode, a hard-wired analog logic circuit, software stored on a computer-readable medium or signal, an optical computing device, a networked system of electronic and/or optical devices, a special purpose computing device, an integrated circuit device, a semiconductor chip, and a software module or object stored on a computer-readable medium or signal, for example.

Embodiments of the method and system (or their sub-components or modules), may be implemented on a general-purpose computer, a special-purpose computer, a programmed microprocessor or microcontroller and peripheral integrated circuit element, an ASIC or other integrated circuit, a digital signal processor, a hardwired electronic or logic circuit such as a discrete element circuit, a programmed logic circuit such as a programmable logic device (PLD), programmable logic array (PLA), field-programmable gate array (FPGA), programmable array logic (PAL) device, or the like. In general, any process capable of implementing the functions or steps described herein can be used to implement embodiments of the method, system, or a computer program product (software program stored on a non-transitory computer readable medium).

Furthermore, embodiments of the disclosed method, system, and computer program product may be readily implemented, fully or partially, in software using, for example, object or object-oriented software development environments that provide portable source code that can be used on a variety of computer platforms. Alternatively, embodiments of the disclosed method, system, and computer program product can be implemented partially or fully in hardware using, for example, standard logic circuits or a very-large-scale integration (VLSI) design. Other hardware or software can be used to implement embodiments depending on the speed and/or efficiency requirements of the systems, the particular function, and/or particular software or hardware system, microprocessor, or microcomputer being utilized.

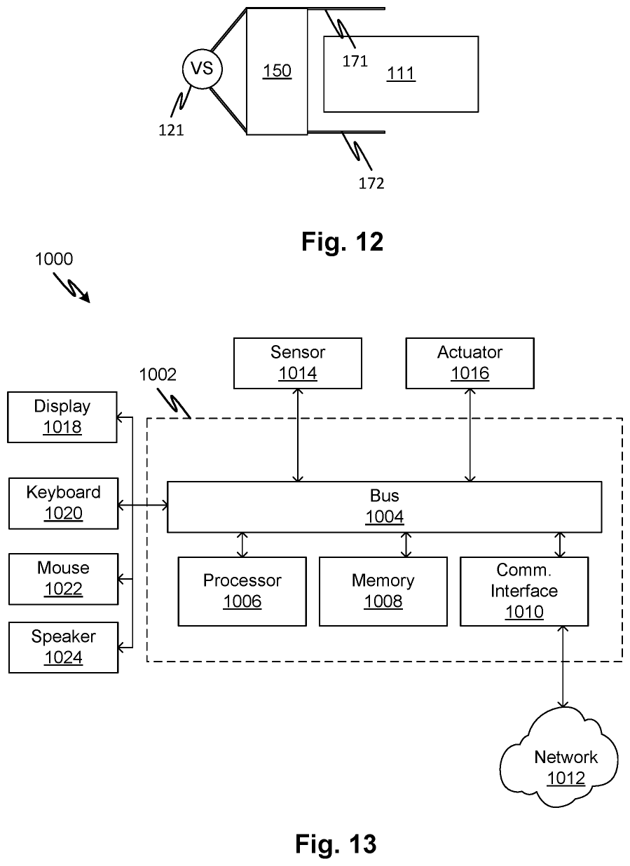

FIG. 13 shows a block diagram of an example computer system according to embodiments of the disclosed subject matter. FIG. 13 shows a disclosure of a computer system that embodies elements of any controllers disclosed herein. In various embodiments, all or parts of system 1000 may be included in a pollution treatment device/system. In these embodiments, all or parts of system 1000 may provide the functionality of a controller of the device or system. In some embodiments, all or parts of system 1000 may be implemented as a distributed system, for example, as a cloud-based system.

System 1000 includes a computer 1002 such as a personal computer or workstation or other such computing system that includes a processor 1006. However, alternative embodiments may implement more than one processor and/or one or more microprocessors, microcontroller devices, or control logic including integrated circuits such as ASIC.

Computer 1002 further includes a bus 1004 that provides communication functionality among various modules of computer 1002. For example, bus 1004 may allow for communicating information/data between processor 1006 and a memory 1008 of computer 1002 so that processor 1006 may retrieve stored data from memory 1008 and/or execute instructions stored on memory 1008. In one embodiment, such instructions may be compiled from source code/objects provided in accordance with a programming language such as Java, C++, C#, .net, Visual Basic™ language, LabVIEW, or another structured or object-oriented programming language. In one embodiment, the instructions include software modules that, when executed by processor 1006, provide cooking pollutant control functionality according to any of the embodiments disclosed herein.

Memory 1008 may include any volatile or non-volatile computer-readable memory that can be read by computer 1002. For example, memory 1008 may include a non-transitory computer-readable medium such as ROM, PROM, EEPROM, RAM, flash memory, disk drive, etc. Memory 1008 may be a removable or non-removable medium.

Bus 1004 may further allow for communication between computer 1002 and a display 1018, a keyboard 1020, a mouse 1022, and a speaker 1024, each providing respective functionality in accordance with various embodiments disclosed herein.

Computer 1002 may also implement a communication interface 1010 to communicate with a network 1012 to provide any functionality disclosed herein, for example, for alerting that a filter element is depleted or is close to being depleted. Communication interface 1010 may be any such interface known in the art to provide wireless and/or wired communication, such as a network card or a modem.

Bus 1004 may further allow for communication with one or more sensors 1014 and one or more actuators 1016, each providing respective functionality in accordance with various embodiments disclosed herein, for example, for measuring signals.

It is, thus, apparent that there is provided, in accordance with the present disclosure, a filtration system. Many alternatives, modifications, and variations are enabled by the present disclosure. Features of the disclosed embodiments can be combined, rearranged, omitted, etc., within the scope of the invention to produce additional embodiments. Furthermore, certain features may sometimes be used to advantage without a corresponding use of other features. Accordingly, Applicants intend to embrace all such alternatives, modifications, equivalents, and variations that are within the spirit and scope of the present invention. Moreover, embodiments of the disclosed method, system, and computer program product can be implemented in software executed on a programmed general-purpose computer, a special purpose computer, a microprocessor, or the like.

1. (canceled)

2. The system of claim 19, wherein the odor removal stage includes a carbon filter.

3. The system of claim 2, wherein the odor removal stage also includes an ultraviolet light source.

4. The system of claim 19 wherein the particulate removal stage includes a pocket filter.

5. The system of claim 19 wherein the particulate removal stage includes an electrostatic precipitator filter.

6. The system of claim 2, further comprising:
a controller that receives signals from the VOC sensor and uses the signals to generate data indicative of a remaining life of the carbon filter.

7-18. (canceled)

19. An odor removal system, comprising:
an exhaust hood configured to capture fumes from a cooking appliance;
the exhaust hood conveying fumes to a particulate removal stage which conveys the fumes to an odor removal stage that includes an odor removal filter; and
the odor removal filter having a volatile organic compound (VOC) sensor with a sampling device having a first sampling port and a second sampling port configured to convey samples of fumes from upstream and downstream of the odor removal filter to the VOC sensor.

20. The system of claim 19, wherein the sampling device conveys the samples of fumes intermittently to the VOC sensor in order to obtain signals from different locations from a same VOC sensor.

21-36. (canceled)

37. A method of estimating a remaining life of a filter in a flow path, comprising:
providing a first sensing location along the flow path upstream of the filter;
providing a second sensing location along the flow path downstream of the filter;
detecting a quality of air at the first sensing location;
detecting the quality of air at the second sensing location;
comparing the detected quality of air from the first sensing location with the detected quality of air from the second sensing location; and
outputting a measure of the remaining life of the filter based on a result of the comparing.

38. The method of claim 37, further comprising:
providing a first volatile organic compound sensor in fluid communication with the first sensing location through a first sampling port and in fluid communication with the second sensing location through a second sampling port,
wherein
the detecting the quality of air at the first sensing location includes outputting a first signal from the first volatile organic compound sensor, and
the detecting the quality of air at the second sensing location includes outputting a second signal from the first volatile organic compound sensor.

39. The method of claim 38, wherein
the comparing the detected quality of air includes providing the first signal and the second signal to a device.

40. The method of claim 39, wherein
the device is one of a full adder, a half adder, a full subtractor, a half subtractor, and an analog circuit that includes an operational amplifier.

41. The method of claim 39, wherein
the device is a digital controller.

42. The method of claim 40, wherein
the device outputs an output signal that represents the remaining life of the filter.

43. The method of claim 42, wherein
a voltage level of the output signal represents the remaining life of the filter.

44. The method of claim 42, wherein
the output signal represents the remaining life of the filter as a digital signal.

45. The method claim 41, wherein
the device outputs an output signal that represents the remaining life of the filter.

46. The method of claim 45, wherein
a voltage level of the output signal represents the remaining life of the filter.

47. The method of claim 45, wherein
the output signal represents the remaining life of the filter as a digital signal.
